(19) United States
(12) Patent Application Publication
(10) Pub. No.: US 2022/0313693 A1
(43) Pub. Date: Oct. 6, 2022

(54) METHODS OF TREATING HIV IN PEDIATRIC PATIENTS WITH RILPIVIRINE

(71) Applicant: Janssen Sciences Ireland Unlimited Company, Co. Cork (IE)

(72) Inventors: Herta CRAUWELS, Wilrijk (BE); Simon VANVEGGEL, Merksem (BE); Veerie VAN EYGEN, Itegem (BE)

(21) Appl. No.: 17/621,759

(22) PCT Filed: Jul. 3, 2020
(86) PCT No.: PCT/EP2020/068913

§ 371 Date: Dec. 22, 2021

Related U.S. Application Data
(60) Provisional application No. 62/870,413, filed on Jul. 3, 2019.

(51) Int. Cl. A61K 31/505; A61P 31/18

(52) U.S. Cl. CPC A61K 31/505; A61P 31/18

(57) ABSTRACT

The disclosure is directed to the use of rilpivirine, or a salt thereof, to treat HIV infection in pediatric subjects.

### TECHNICAL FIELD

The disclosure is directed to the use of rilpivirine, or a salt thereof, to treat HIV infections in pediatric subjects.

### BACKGROUND

Subjects infected with HIV are routinely treated with combinations of multiple drugs (highly active antiretroviral [ARV] therapy) including nucleoside/nucleotide reverse transcriptase inhibitors (N[t]RTIs), non-nucleoside reverse transcriptase inhibitors (NNRTIs), protease inhibitors, pharmacokinetic (PK) boosters, integrase inhibitors, and fusion inhibitors. This treatment reduces HIV-1 ribonucleic acid (RNA) to undetectable levels in a substantial proportion of subjects and counteracts the risk of viral resistance development.

Rilpivirine (RPV, formerly known as TMC278 [R278474]), a diarylpyrimidine derivative, is a potent non nucleoside reverse transcriptase inhibitor (NNRTI) with in vitro activity against wild type (WT) HIV 1 and against NNRTI resistant HIV 1 mutants. A medical need still exists for the development of age/weight-appropriate therapies in adolescents and children.

### SUMMARY

The disclosure is directed to methods of treating pediatric subjects infected with an HIV virus. The subjects weigh 11 kg or more, and treatment experienced, and were administered a first antiretroviral regimen that has been discontinued. The methods comprise administration of 25 mg or less of a non-nucleoside reverse transcriptase inhibitor that is rilpivirine (or an equivalent amount of a pharmaceutically acceptable salt of rilpivirine), once daily. According to the described methods, the subject will exhibit a viral load of less than or equal to 50 copies of HIV virus particles per mL, of blood plasma (≤50 c/mL) after at least 24 week of the once-daily administration of the rilpivirine, or equivalent amount of the pharmaceutically acceptable salt of rilpivirine.

The disclosure is directed to the use of rilpivirine or a pharmaceutically acceptable salt of rilpivirine for the manufacture of a medicament for the treatment of pediatric subjects infected with an HIV virus. The disclosure is directed to rilpivirine or a pharmaceutically acceptable salt of rilpivirine for use in the treatment of pediatric subjects infected with an HIV virus. The subjects weigh 11 kg or more, and are treatment experienced, and were administered a first antiretroviral regimen that has been discontinued. The uses comprise administration of 25 mg or less of a non-nucleoside reverse transcriptase inhibitor that is rilpivirine (or an equivalent amount of a pharmaceutically acceptable salt of rilpivirine), once daily. According to the described uses, the subject will exhibit a viral load of less than or equal to 50 copies of HIV virus particles per mL, of blood plasma (≤50 c/mL) after at least 24 week of the once-daily administration of the rilpivirine, or equivalent amount of the pharmaceutically acceptable salt of rilpivirine.

The disclosure is directed to methods of treating pediatric subjects infected with an HIV virus. The subjects weigh 11 kg or more, and are antiretroviral-naïve HIV-1 infected pediatric subjects, in particular adolescents and children aged ≥6 to <18 years. The methods comprise administration of 25 mg or less of a non-nucleoside reverse transcriptase inhibitor that is rilpivirine (or an equivalent amount of a pharmaceutically acceptable salt of rilpivirine), once daily. According to the described methods, the subject will exhibit a viral load of less than or equal to 50 copies of HIV virus particles per mL, of blood plasma (≤50 c/mL) after at least 24 week of the once-daily administration of the rilpivirine, or equivalent amount of the pharmaceutically acceptable salt of rilpivirine.

The disclosure is directed to the use of rilpivirine or a pharmaceutically acceptable salt of rilpivirine for the manufacture of a medicament for the treatment of pediatric subjects infected with an HIV virus. The disclosure is directed to rilpivirine or a pharmaceutically acceptable salt of rilpivirine for use in the treatment of pediatric subjects infected with an HIV virus. The subjects weigh 11 kg or more, and are antiretroviral-naïve HIV-1 infected pediatric subjects, in particular adolescents and children aged ≤6 to <18 years. The uses comprise administration of 25 mg or less of a non-nucleoside reverse transcriptase inhibitor that is rilpivirine or an equivalent amount of a pharmaceutically acceptable salt of rilpivirine, once daily. According to the described uses, the subject will exhibit a viral load of less than or equal to 50 copies of HIV virus particles per mL, of blood plasma (≤50 c/mL) after at least 24 week of the once-daily administration of the rilpivirine, or equivalent amount of the pharmaceutically acceptable salt of rilpivirine.

### DETAILED DESCRIPTION OF ILLUSTRATIVE EMBODIMENTS

The present disclosure may be understood more readily by reference to the following detailed description taken in connection with the accompanying examples, which form a part of this disclosure. It is to be understood that this disclosure is not limited to the specific devices, methods, applications, conditions or parameters described and/or shown herein, and that the terminology used herein is for the purpose of describing particular embodiments by way of example only and is not intended to be limiting of the claimed disclosure.

As used in the specification including the appended claims, the singular forms “a,” “an,” and “the” include the plural, and reference to a particular numerical value includes at least that particular value, unless the context clearly dictates otherwise.

When a range of values is expressed, another embodiment includes from the one particular value and/or to the other particular value. All ranges are inclusive and combinable. Further, reference to values stated in ranges include each and every value within that range. When values are expressed as approximations, by use of the antecedent “about,” it will be understood that the particular value forms another embodiment. The term “about” as used herein when referring to a measurable value such as an amount, a temporal duration, and the like, is meant to encompass reasonable variations of the value, such as, for example, ±10% from the specified value. For example, the phrase “about 50%” can include ±10% of 50, or from 45% to 55%.

It is to be appreciated that certain features of the disclosure which are, for clarity, described herein in the context of separate embodiments, may also be provided in combination in a single embodiment. Conversely, various features of the disclosure that are, for brevity, described in the context of a single embodiment, may also be provided separately or in any subcombination.

The present invention may be understood by reference to the following detailed description which forms a part of this disclosure. The invention is not limited to the specific methods, conditions or parameters described and/or shown herein, and the terminology used herein is for the purpose of describing particular embodiments by way of example only and is not intended to be limiting of the claimed invention.

Scientific and technical terms used in connection with the present application shall have the meanings that are commonly understood by those of ordinary skill in the art, unless otherwise defined herein.

“Pharmaceutically acceptable salt” refers to a salt of a compound of the disclosure that is pharmaceutically acceptable and that possesses the desired pharmacological activity of the parent compound. In particular, such salts are non-toxic may be inorganic or organic acid addition salts and base addition salts. Specifically, such salts include: (1) acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, ethanesulfonic acid, 1,2-ethane-disulfonic acid, 2-hydroxy-ethanesulfonic acid, benzenesulfonic acid, 4-chlorobenzenesulfonic acid, 2-naphthalene-sulfonic acid, 4-toluenesulfonic acid, camphorsulfonic acid, 4-methylbicyclo[2.2.2]-oct-2-ene-1-carboxylic acid, glucoheptonic acid, 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; or (2) salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, N-methylglucamine and the like. Salts further include, by way of example only, sodium, potassium, calcium, magnesium, ammonium, tetraalkylammonium, and the like; and when the compound contains a basic functionality, salts of non-toxic organic or inorganic acids, such as hydrochloride, hydrobromide, tartrate, mesylate, acetate, maleate, oxalate and the like.

Rilpivirine at a dose of 25 mg once daily has been approved for treatment of antiretroviral (ARV) treatment naive HIV 1 infected adults in multiple countries, including the United States, Canada, Japan, and countries in the European Union, either as a single-agent 25-mg tablet (EDURANT) or as part of several fixed dose combinations (ie, with the integrase inhibitor dolutegravir [DTG], with tenofovir disoproxil fumarate/emtricitabine [TDF/FTC], and with tenofovir alafenamide/FTC [TAF/FTC]). Brand names of rilpivirine-containing products include COMPLERA (emtricitabine/rilpiriring/tenofovir disoproxil fumarate), ODEFSEY, and JULUCA (dolutegravir/rilpivirine).

The disclosure is directed to methods of or used for treating a pediatric subject infected with an HIV virus. In preferred aspects, the pediatric subject is infected with an HIV-1 virus. The pediatric subjects will be less than 18 years old, preferably ≥2 years to <12 years. In other aspects, the pediatric subject is ≥6 years to <12 years. In some aspects, the pediatric subject is 1, 2, 3, 4, 5, 6, 7, 8, 9, 10, 11, 12, 13, 14, 15, 16, or 17 years old.

According to the disclosure, the pediatric subjects treated using the described methods or uses weigh 11 kg or more. In some aspects, the pediatric subjects treated using the described methods or uses weigh 11 kg to 25 kg, for example, 11, 12, 13, 14, 15, 16, 17, 18, 19, 20, 21, 22, 23, 24, or 25 kg. In other aspects, the pediatric subjects treated using the described methods weigh more than 25 kg, for example 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, or 70 kg.

According to the disclosure, the pediatric subjects are “treatment experienced,” that is, the subjects have previously been administered antiretroviral drugs. Also according to the disclosure, the pediatric subjects have been previously administered a first (e.g., a prior) anti-retroviral regimen of one or more anti-retroviral drugs, and that first antiretroviral regimen has been discontinued prior to the initiation of any of the methods disclosed herein. In some aspects, the first antiretroviral regimen is discontinued 12 hours or more, prior to the initiation of any method disclosed herein.

According to an aspect, the pediatric subjects are “treatment naïve”, that is, the subjects have previously not been administered antiretroviral drugs.

According to the described methods or uses, the pediatric subject (preferably ≥2 years to <12 years) is administered about 25 mg or less, preferably 25 mg or less, of a non-nucleoside reverse transcriptase inhibitor that is rilpivirine. Preferably, in the methods or uses of the disclosure, the only non-nucleoside reverse transcriptase inhibitor administered to the pediatric subjects is rilpivirine or a salt thereof. In some aspects, the pediatric subject (preferably ≥2 years to <12 years) is administered a pharmaceutically acceptable salt of rilpivirine in an amount that is equivalent to 25 mg or less of rilpivirine. Rilpivirine salts include, for example, rilpivirine hydrochloride.

In some aspects, the pediatric subject weighs 11 kg to 25 kg. In some of these aspects, the pediatric subject is administered 15 mg of rilpivirine (or an equivalent amount of a pharmaceutically acceptable salt of rilpivirine), once daily.

In some aspects, the pediatric subject weighs 11 kg to 25 kg (11 kg to <25 kg). In some of these aspects, the pediatric subject is administered 15 mg of rilpivirine (or an equivalent amount of a pharmaceutically acceptable salt of rilpivirine), once daily. In some aspects the pediatric subject is treatment-naïve. In some aspects, the pediatric subject is treatment experienced.

In some aspects, the pediatric subject weighs more than 25 kg. In some of these aspects, the pediatric subject is administered 25 mg of rilpivirine (or an equivalent amount of a pharmaceutically acceptable salt of rilpivirine), once daily.

In some aspects, the pediatric subject weighs 25 kg or more (≥25 kg). In some of these aspects, the pediatric subject is administered 25 mg of rilpivirine (or an equivalent amount of a pharmaceutically acceptable salt of rilpivirine), once daily. In some aspects the pediatric subject is treatment-naïve. In some aspects, the pediatric subject is treatment experienced.

In some aspects, the pediatric subject is administered 2.5 mg to about 25 mg, for example, 2.5, 5, 7.5, 10, 12.5, 15, 17.5, 20, 22.5, or 25 mg of rilpivirine (or an equivalent amount of a pharmaceutically acceptable salt of rilpivirine), once daily.

In some aspects, the amount of rilpivirine or pharmaceutically acceptable salt thereof is administered as a single unit dosage form. That is, the entirety of the daily amount of the rilpivirine or pharmaceutically acceptable salt thereof is administered in a single dose that is a tablet or capsule. For example, the entirety of the daily amount of the rilpivirine is administered as a 25 mg tablet or capsule or a 15 mg tablet or capsule.

In some aspects, the amount of rilpivirine or pharmaceutically acceptable salt thereof is administered in multiple unit dosage forms. That is, the entirety of the daily amount of the rilpivirine or pharmaceutically acceptable salt thereof is administered as several dosage units. For example, a 15 mg daily rilpivirine dose is administered as six tablets or capsules, each tablet or capsule containing 2.5 mg of rilpivirine. In another example, the 17.5 mg daily rilpivirine dose is administered as seven tablets or capsules, each tablet or capsule containing 2.5 mg of rilpivirine. In another example, the 7.5 mg daily rilpivirine dose is administered as three tablets or capsules, each tablet or capsule containing 2.5 mg of rilpivirine.

In some aspects of the disclosure, the rilpivirine administration (or the administration of the equivalent amount of a pharamceutically acceptable salt of rilpiririne) is when the subject is in a fed state. In some aspects of the disclosure, the rilpivirine administration (or the administration of the equivalent amount of a pharamceutically acceptable salt of rilpiririne) is when the subject is in a fasted state.

According to the described methods, the pediatric subject exhibits a viral load of less than or equal to 50 copies of HIV virus particles (e.g., HIV-1 virus particles) per mL of blood plasma after at least 24 weeks of the once-daily rilpivirine (or rilpivirine salt) administration.

In some aspects of the disclosure, the pediatric subject exhibits a viral load of less than or equal to 50 copies of HIV virus particles (e.g., HIV-1 virus particles) per mL of blood plasma after at least 48 weeks of the once-daily rilpivirine (or rilpivirine salt) administration.

In some aspects of the disclosure, the pediatric subject exhibits a viral load of less than or equal to 50 copies of HIV virus particles (e.g., HIV-1 virus particles) per mL of blood plasma after between 24 and 48 weeks (e.g., 24, 25, 26, 27, 28, 29, 30, 31, 32, 33, 34, 35, 36, 37, 38, 39, 40, 41, 42, 43, 44, 45, 46, 47, or 48 weeks) of the once-daily rilpivirine (or rilpivirine salt) administration.

In some aspects, in addition to the rilpivirine or rilpivirine salt administration, the pediatric subjects will be administered an antiretroviral (ARV) background regimen that includes one or more drugs that is not rilpivirine or a rilpivirine salt. The ARV background regimen can include any one or more active pharmaceutical ingredients (APIs) used in the art to treat subjects infected with an HIV virus, for example, an HIV-1 virus. APIs useful in treating subjects infected with an HIV virus include nucleoside reverse transcriptase inhibitors and nucleotide reverse transcriptase inhibitors. In some aspects, the ARV background regimen includes two, or more than two, nucleoside reverse transcriptase inhibitors and/or nucleotide reverse transcriptase inhibitors. Examples of APIs for use in the ARV background regimen include, for example, azidothymidine (AZT), abacavir (ABC), lamivudine (3TC), dolutegravir, tenofovir, a pharmaceutically acceptable salt of tenofovir, a tenofovir prodrug (e.g., tenofovir disoproxil, tenofovir alafenamide), a pharmaceutically acceptable salt of a tenofovir prodrug (e.g., tenofovir disoproxil fumarate, tenofovir alafenamide fumarate), emtricitabine, and combinations thereof.

In some aspects of the disclosure, the pediatric subject is virally supressed prior to the administration of the rilpivirine or pharmaceutically acceptable salt of rilpivirine. For example, the pediatric subject may exhibit a viral load of less than or equal to 50 copies of HIV virus particles (e.g., HIV-1 virus particles) per mL of blood plasma prior to the once-daily administration of the rilpivirine or pharmaceutically acceptable salt of rilpivirine. In some aspects, the pediatric subject has been virally supressed for at least 12 months, prior to the once-daily administration of the rilpivirine or pharmaceutically acceptable salt of rilpivirine. In some aspects, the pediatric subject has been virally supressed for 2 to 12 months, prior to the once-daily administration of the rilpivirine or pharmaceutically acceptable salt of rilpivirine. In some aspects, the pediatric subject has been virally supressed for 1, 2, 3, 4, 5, 6, 7, 8, 9, 10, 11, or 12 months, prior to the once-daily administration of the rilpivirine or pharmaceutically acceptable salt of rilpivirine.

In some aspects of the disclosure, the pediatric subject is not virally suppressed prior to the administration of the rilpivirine or pharmaceutically acceptable salt of rilpivirine. In some aspects of the disclosure, the pediatric subject is treatment naïve prior to the administration of the rilpivirine or pharmaceutically acceptable salt of rilpivirine.

In some aspects of the disclosure, the methods of or uses for treating the pediatric subjects do not significantly affect pubertal development, for example, pubertal development as assessed by Tanner staging. In other aspects of the disclosure, the methods of or uses for treating the pediatric subjects do not significantly affect adolescent growth.

In some aspects of the disclosure, the methods or uses result in a lower incidence of Grade 3 or 4 adverse drug reactions (ADRs), as compared to prior treatment methods. ADRs include, for example, headache, nausea, insomnia, dizziness, abnormal dreams, rash, abdominal pain, depression, fatigue, and vomiting.

In some aspects of the disclosure, the methods or uses result in a lower incidence of Grade 2 ADRs, as compared to prior treatment methods. ADRs include, for example, depression, headache, insomnia, transaminases increased, rash, and abdominal pain.

In some aspects of the disclosure, the methods or uses result in a lower incidence of virologic failure, as compared to prior treatment methods. In some aspects of the disclosure, the methods or uses result in a lower incidence of treatment resistance, as compared to prior treatment methods. In some aspects of the disclosure, the methods or uses result in a lower incidence of drug-drug interactions, as compared to prior treatment methods. In some aspects of the disclosure, the methods or uses result in a lower incidence of body fat redistribution and/or body fat accumulations (e.g., central obesity, dorsocervical fat enlargement (buffalo hump), peripheral wasting, facial wasting, breast enlargement, and ‘cushingoid appearance’), as compared to prior treatment methods. In some aspects of the disclosure, the methods or uses result in a lower incidence of immune reconstitution inflammatory syndrome (e.g., inflammatory response to indolent or residual opportunistic infections (such as Mycobacterium avium complex, cytomegalovirus, Pneumocystis jiroveci pneumonia, and tuberculosis)), as compared to prior treatment methods.

In some aspects, the disclosure is directed to the use of rilpivirine or a pharmaceutically acceptable salt of rilpivirine for the manufacture of a medicament for the treatment of a pediatric subject infected with an HIV virus, wherein the pediatric subject weighs 11 kg to 25 kg (11 kg to <25 kg), wherein the pediatric subject is administered or is to be administered 15 mg of rilpivirine (or an equivalent amount of a pharmaceutically acceptable salt of rilpivirine, in particular rilpivirine hydrochloride), once daily, and wherein the pediatric subject is treatment-naïve. In some aspects, the pediatric subject is treatment experienced.

In some aspects, the disclosure is directed to rilpivirine or a pharmaceutically acceptable salt of rilpivirine for use in the treatment of a pediatric subject infected with an HIV virus, wherein the pediatric subject weighs 11 kg to 25 kg (11 kg to <25 kg), wherein the pediatric subject is administered or is to be administered 15 mg of rilpivirine (or an equivalent amount of a pharmaceutically acceptable salt of rilpivirine, in particular rilpivirine hydrochloride), once daily, and wherein the pediatric subject is treatment-naïve. In some aspects, the pediatric subject is treatment experienced.

In some aspects, the disclosure is directed to the use of rilpivirine or a pharmaceutically acceptable salt of rilpivirine for the manufacture of a medicament for the treatment of a pediatric subject infected with an HIV virus, wherein the pediatric subject weighs 25 kg or more (≥25 kg), wherein the pediatric subject is administered or is to be administered 25 mg of rilpivirine (or an equivalent amount of a pharmaceutically acceptable salt of rilpivirine, in particular rilpivirine hydrochloride), once daily, and wherein the pediatric subject is treatment-naïve. In some aspects, the pediatric subject is treatment experienced.

In some aspects, the disclosure is directed to rilpivirine or a pharmaceutically acceptable salt of rilpivirine for use in the treatment of a pediatric subject infected with an HIV virus, wherein the pediatric subject weighs 25 kg or more (≥25 kg), wherein the pediatric subject is administered or is to be administered 25 mg of rilpivirine (or an equivalent amount of a pharmaceutically acceptable salt of rilpivirine, in particular rilpivirine hydrochloride), once daily, and wherein the pediatric subject is treatment-naïve. In some aspects, the pediatric subject is treatment experienced.

The following examples of illustrative of the inventions and are not intended to be limiting.

### EXAMPLES

This is a Phase 2, open-label, single-arm, multicenter, interventional study in HIV 1 infected participants (boys and girls) aged ≥2 to <12 years with a body weight of at least 11 kg to evaluate the PK, safety, tolerability, and efficacy of switching to RPV once daily in combination with other, investigator selected ARVs.

All participants will have a screening phase to be completed within 6 weeks. The screening phase can be prolonged with maximum 2 weeks in case of unforeseeable circumstances. All participants will receive open-label treatment for 48 weeks in the study intervention phase. The total study duration for each participant, including screening and study intervention phases, will be approximately 54 weeks. An Independent Data Monitoring Committee (IDMC) will be commissioned for this study.

### Intervention Groups and Duration

Rilpivirine (25 mg or a weight-based dose, or an equivalent amount of a rilpivirine salt) will be orally administered once daily in combination with an investigator-selected background regimen containing other ARVs such as N(t)RTIs and integrase inhibitors. Protease inhibitors and ARVs requiring a PK booster, however, are disallowed from baseline onwards

The participants will continue the study intervention and ARV background regimen (through the data review periods, if applicable) until they all reach a total treatment duration of 48 weeks (or discontinue earlier). Dose adjustments of RPV due to changes in body weight, if applicable, are allowed.

### Efficacy Evaluations

Key efficacy assessments include determination of plasma HIV-1 RNA viral load and measurement of CD4+ cell count.

### Pharmacokinetic Evaluations

### Pharmacokinetics

Based on the individual plasma concentration-time data, using the actual dose taken and the actual PK sampling times, the following PK parameters of RPV will be derived:

C₀ₕ, Cₘᵢₙ, Cₘₐₓ, C_{ss,av}, tₘₐₓ, AUC₂₄ₕ, CL/F, Vₛₛ/F, and FI.

### Population Pharmacokinetics

Based on the individual plasma concentration-time data, using the actual dose taken and the actual PK sampling time, PK parameters and exposure information of RPV will be derived using population PK modeling.

### Pharmacokinetic/Pharmacodynamic Evaluations

Pharmacokinetic/PD evaluations will be performed to study the relationship between PK and safety/efficacy variables.

### Safety Evaluations

Key safety assessments will include the monitoring of (S)AEs and HIV-related events (including AIDS-defining illnesses and Stage-3-defining Opportunistic Illnesses in HIV Infection [cut-off for Stage-3 illnesses is 6 years of age per criteria from 2014]), clinical laboratory tests (including endocrine assessments in participants aged ≥6 to <12 years), cardiovascular safety monitoring (vital signs and 12-lead ECGs), and physical examination (including growth). In addition, an evaluation of depression will be performed using questionnaires or other means (as available at the site) as part of local standard of care for this population.

### Other Evaluations

Other assessments and procedures include resistance testing through HIV-1 genotyping and a retrospective evaluation of RAMs in PBMCs, documenting RPV intake through diary completion, treatment adherence, and

### Statistical Methods

The primary analysis (with formal database lock) will be done when all participants have reached Week 24 (or discontinued earlier). The final analysis (with formal database lock) will be done when all participants have reached Week 48 (or discontinued earlier). A detailed Statistical Analysis Plan (SAP) for each analysis will be written and signed off prior to database lock.

### Efficacy Analyses

### Plasma Viral Load

An outcome analysis (ie, proportion of participants with a plasma viral load <50 and <400 HIV-1 RNA copies/mL) will be performed using Snapshot approach. The Snapshot analysis is based on the last observed plasma viral load data within the visit window (ie, Weeks 24 and 48). The proportion of participants with virologic failure (ie, HIV-1 RNA ≥50 and ≥400 copies/mL) per Snapshot approach will be provided. Participants who switched ARVs for tolerability reasons not allowed per protocol will be considered as virologic failures for this Snapshot approach. Proportions will be expressed as percentages with Clopper Pearson 95% confidence interval (CI) at each time point.

Time-to-event data (ie, time to loss of virologic response) will be graphically presented by means of Kaplan-Meier curves.

### CD4⁺ Cell Count

The analysis will be based on observed values and on imputed values using NC=F, ie, participants who prematurely discontinued the study will have their CD4⁺ cell count following discontinuation imputed with the baseline value (resulting in a change of 0), and will have last-observation-carried-forward imputation for intermediate missing values.

Actual data and changes from baseline will be descriptively and graphically presented.

### Safety Analysis

### Adverse Events/HIV-Related Events

For each treatment-emergent AE/HIV-related event, the percentage of participants who experience at least 1 occurrence of the given event will be tabulated per study phase (ie, screening phase, intervention phase, and follow-up). Separate tabulations will be made by severity and relationship to the study intervention, as appropriate.

Summaries, listings, datasets, or participant narratives may be provided, as appropriate, for those participants who die, who discontinue study intervention due to an AE, or who experience a grade 3/4 AE, an AE of special interest, or an SAE.

### Clinical Laboratory Tests

Laboratory data will be summarized by type of laboratory test. Descriptive statistics will be calculated for each laboratory analyte at baseline and for observed values and changes from baseline at each scheduled time point. Descriptive statistics include number of observations (n), mean, standard deviation (SD), median, minimum, and maximum.

Frequency tabulations of the changes from baseline will be presented in pre- versus post-intervention cross-tabulations (with classes for below, within, and above normal ranges). For the tests available, laboratory abnormalities will be determined using the Division of AIDS (DAIDS) grading table. Frequency tabulations of worst abnormality grade after baseline will be generated. As appropriate, frequency tabulations and listings will be provided for participants who develop a grade 3/4 laboratory abnormality.

### Electrocardiogram

Descriptive statistics of ECG values and changes from baseline will be summarized at each scheduled time point. Descriptive statistics include number of observations (n), mean, SD, median, minimum, and maximum. Frequency tabulations of the abnormalities will be made.

### Vital Signs

Descriptive statistics of pulse rate and blood pressure (systolic and diastolic) (supine and standing) values and changes from baseline will be summarized at each scheduled time point. Descriptive statistics include number of observations (n), mean, SD, median, minimum, and maximum. The percentage of participants with values beyond clinically important limits will be summarized at each time point.

### Physical Examination

Physical examination findings will be summarized at each scheduled time point per body system. Physical examination abnormalities will be listed.

Growth will be followed regularly and evaluated consistently using standardized growth charts. Descriptive statistics of height, height-for-age, weight, weight-for-age, body mass index (BMI), and BMI-for-age will be calculated at baseline and for observed values and changes from baseline at each scheduled time point. Descriptive statistics include number of observations (n), mean, SD, median, minimum, and maximum.

Tanner stage (for pubic hair and genitalia/breasts) will be cross-tabulated versus baseline by age. In addition, in girls, the occurrence of first menses during treatment will also be cross-tabulated versus baseline, and the date of menarche will be listed.

### Pharmacokinetic Analysis

Descriptive statistics, including same size (n), arithmetic mean, SD, (percentage of) coefficient of variation ([%]CV), geometric mean, median, minimum, and maximum, will be calculated for all individual derived PK parameters of RPV.

Efficacy and safety parameters will be subjected to a PK/PD analysis. Various efficacy and safety parameters will be linked to the PK of RPV applying graphical tools and, if feasible, statistical models.

### Other Analyses

Frequency tabulations and listings will be generated.

### Endpoints

### Primary Endpoints

Area under the plasma concentration-time curve from time of administration up to 24 hours postdose of RPV.

Incidence of grade 3/4 AEs, SAES, HIV-related events (including acquired immune deficiency syndrome [AIDS]-defining illnesses and Stage-3-defining Opportunistic Illnesses in HIV Infection), and AEs leading to discontinuation of study intervention through 24 weeks of study treatment.

### Secondary Endpoints

Incidence and severity of AEs/HIV-related events and their relatedness to RPV through 24 and 48 weeks of study treatment.

Change from baseline over time and shift in toxicity grades/abnormalities versus reference for clinical laboratory parameters, ECG parameters, vital signs, and physical examination through 24 and 48 weeks of study treatment.

Proportion of participants with HIV-1 RNA <50 and ≥50 copies/mL using the Food and Drug Administration (FDA) Snapshot approach through 24 and 48 weeks of study treatment.

Immunologic changes, measured by CD4⁺ cell count (absolute and percentage relative to total lymphocytes), through 24 and 48 weeks of study treatment.

Pharmacokinetic parameters of RPV (other than area under the plasma concentration-time curve [AUC]).

Pharmacokinetic parameters of RPV, as derived by population PK modeling, through 24 and 48 weeks of study treatment.

Viral genotype at the time of virologic failure through 24 and 48 weeks of study treatment.

Treatment adherence, as assessed by the Pediatric European Network for the Treatment of AIDS (PENTA) adherence questionnaire and by study intervention accountability, through 24 and 48 weeks of study treatment.

Mutations in HIV-1 DNA or in HIV-1 RNA, as assessed by retrospective peripheral blood mononuclear cell (PBMC)- or plasma-based analyses, through 24 and 48 weeks of study treatment.

### Study Design

This is a Phase 2, open-label, single-arm, multicenter, interventional study in HIV-1-infected participants (boys and girls) aged ≥2 to <12 years with a body weight of at least 11 kg to evaluate the PK, safety, tolerability, and efficacy of switching to RPV once daily in combination with other, investigator-selected ARVs.

To comply with overall regulatory requirements, approximately 40 participants (including approximately 12 participants with a body weight of <25 kg at baseline) will be enrolled in this study. A target of approximately 25 to 30 participants will be enrolled in this study. The actual number of participants in this study will depend on the number of participants enrolled. The participants with a body weight of <25 kg and ≥25 kg will be enrolled in parallel.

Each participant needs to be virologically suppressed (ie, HIV-1 RNA <50 copies/mL) on a stable ARV regimen for at least 6 months at screening and needs to have no history of virologic failure. In addition, the participants should lack any RPV resistance-associated mutations (RAMs) as evidenced by their historical HIV-1 genotyping results, if available. Participants aged ≥2 to <6 years, however, should have historical HIV-1 genotyping results available at screening, to be provided to the sponsor. The availability of the historical HIV-1 genotyping results and the subtype need to be recorded in the CRF. For participants aged ≥6 to <12 years, the availability of historical HIV-1 genotyping results should be recorded in the CRF.

Rilpivirine (25 mg or a weight-based dose, or an equivalent amount of a rilpivirine salt) will be orally administered once daily in combination with an investigator-selected background regimen containing other ARVs such as N(t)RTIs and integrase inhibitors. Protease inhibitors and ARVs requiring a PK booster, however, are disallowed from baseline onwards.

The participants will continue the study intervention and ARV background regimen (through the data review periods, if applicable) until they all reach a total treatment duration of 48 weeks (or discontinue earlier). Dose adjustments of RPV due to changes in body weight, if applicable, are allowed.

All participants will have a screening phase aimed to be completed within 6 weeks. However, the screening phase can be prolonged with maximum 2 weeks in case of unforeseeable circumstances. All participants will receive open-label treatment for 48 weeks in the study intervention phase. Upon study completion, participants who continue to experience clinical benefit from treatment with RPV will be offered the opportunity to continue study treatment. The total study duration for each participant, including screening and study intervention phases, will be approximately 54 weeks.

Key safety assessments will include the monitoring of (S)AEs and HIV-related events (including AIDS-defining illnesses and Stage-3-defining Opportunistic Illnesses in HIV Infection [cut-off for Stage-3 illnesses is 6 years of age per criteria from 2014]), clinical laboratory tests (including endocrine assessments in participants aged ≥6 to <12 years), cardiovascular safety monitoring (vital signs and 12-lead ECGs), and physical examination (including growth). In addition, an evaluation of depression will be performed using questionnaires or other means (as available at the site) as part of local standard of care for this population.

Key efficacy assessments include determination of plasma HIV-1 RNA viral load and measurement of CD4⁺ cell count.

Other assessments and procedures include resistance testing through HIV-1 genotyping and a retrospective evaluation of RAMs in PBMCs, documenting RPV intake through diary completion, treatment adherence, and the like.

The primary analysis (with formal database lock) will be done when all participants have reached Week 24 (or discontinued earlier) followed by a final analysis (with formal database lock) when all participants have reached Week 48 (or discontinued earlier).

### Study Population

Due to the medical need for the development of novel potent ARVs and age/weight-appropriate formulations in children, HIV-1-infected children (boys and girls) aged ≥2 to <12 years will be enrolled.

Children with HIV-infection are known to often present with a stunted growth and a low body weight compared with healthy children, even more so if additional risk factors for growth impairment are present. To ensure that a representative fraction of the HIV-1-infected pediatric population can be studied, children with a body weight as of 11 kg (ie, the 10^{th} percentile of the growth curve for body weight for healthy girls aged 2 years) are allowed to enter the study.

In clinical studies, hypersensitivity reactions have been reported in approximately 5% of adult and pediatric participants receiving abacavir (ABC). Since the risk for developing such reactions has been linked to the presence of the human leukocyte antigen (HLA)-B*5701 allele, participants without prior documented HLA-B*5701 negative results for whom the investigator considers ABC in the background regimen should test negative for HLA-B*5701 at screening to limit the risk of hypersensitivity reactions. If a switch to an ABC-containing background regimen is planned during the study, an HLA-B*5701 test has to be performed to determine eligibility to start ABC treatment (unless prior documented negative results are available).

### Study Intervention Administration

The combined use of multiple ARVs in HIV-1-infected participants is currently recommended due to the inherent high mutation rate of HIV. Therefore, all participants will receive an investigator-selected background regimen in addition to RPV. Consistent with the treatment guidelines for ART, sensitivity to the chosen ARVs will be established at screening using historical HIV-1 genotyping results. Participants aged ≥6 to <12 years are not required to have historical HIV-1 genotyping results available at screening due to limited availability of historical HIV-1 genotyping results for participants in this age group, especially in the developing countries, and the expected fading of HIV-1 mutations.

### Study Assessments

PK assessment will be performed after at least 4 weeks of study treatment. The blood sample collection scheme was designed to accurately and completely describe the PK of RPV with a minimum number of blood samples being collected.

To avoid the accumulation of RAMs and to allow timely study withdrawal, frequent plasma viral load monitoring will be performed in addition to real-time plasma-based viral resistance testing in case of loss of virologic response. Samples for determination of CD4⁺ cell count will be taken in addition to the plasma viral load samples.

Nonclinical studies demonstrated changes in adrenal hormones. As a precaution, clinical laboratory evaluations will also include endocrine assessments in participants aged ≥6 to <12 years to verify whether any clinically relevant adrenal or gonadal effects of RPV are observed.

Delusions and inappropriate behavior have been reported in participants receiving licensed NNRTIs, predominantly in participants with a history of mental illness or substance abuse. To assess the risk of depression in participants treated with RPV, an evaluation will be done using questionnaires or other means (as available at the site) as part of local standard of care for this population. This will determine who needs to be referred for a complete mental health assessment by a mental health professional. Any clinically relevant changes occurring during the study will be reported as AEs.

Drug adherence is critical to the success of any treatment regimen. In addition, in the current study, suboptimal adherence to RPV has an impact on the PK assessments of RPV. Moreover, poor adherence to the background ARV regimen while remaining on only RPV (ie, virtual monotherapy) could not only lead to incomplete suppression of viral replication and treatment failure, but also potentially result in the emergence of a drug-resistant virus. There is evidence that adherence problems occur frequently in children. In a randomized treatment study, caregivers reported that 30% of children missed 1 or more doses of ARVs in the preceding 3 days. These findings illustrate the difficulty of maintaining high adherence levels, and underscore the need to work in partnership with families to make adherence assessment, education, and support integral components of care. In the current study, compliance to RPV and the background ARVs will be assessed by the PENTA adherence questionnaire. Compliance to RPV will also be assessed via pill count (study intervention accountability). If a participant's intake of RPV or background ARVs is not according to the protocol, the investigator will take the necessary measures to ensure future adherence to the protocol.

### Inclusion Criteria

| | |
|---|---|
| 1. | Aged ≥2 to <12 years at screening. |
| 2. | Weighing at least 11 kg at screening. |
| 3. | Have documented chronic HIV-1 infection. |
| 4. | 4.1 Virologically suppressed on a stable ARV regimen with documented evidence |
| | of at least 2 plasma viral loads <50 HIV-1 RNA copies/mL: one within 2-12 months |
| | prior to screening and one at screening |
| 6. | Parent(s) (preferably both if available or as per local requirements) (or the |
| | participant’s legally acceptable representative[s]) must sign an ICF indicating that |
| | he or she understands the purpose of and procedures required for the study and is |
| | willing to allow the child to participate in the study. Assent is also required from |
| | participants capable of understanding the nature of the study (typically aged ≥7 years). |
| 7. | Can comply with the protocol requirements. |
| 8. | Can switch from any ARV class. |
| 9. | Never been treated with a therapeutic HIV vaccine. |
| 10. | Otherwise healthy and medically stable on the basis of physical examination, |
| | medical history, vital signs, and 12-lead ECG performed at screening. If there are |
| | abnormalities, they must be consistent with the underlying illness in the study |
| | population. This determination must be recorded in the participant’s source |
| | documents and initialed by the investigator. |
| 11. | Otherwise healthy on the basis of clinical laboratory tests performed at screening. |
| | If the results of biochemistry, hematology, or urinalysis are outside the normal |
| | reference ranges, the participant may be included only if the investigator judges the |
| | abnormalities or deviations from normal to be not clinically significant or to be |
| | appropriate and reasonable for the study population. This determination must be |
| | recorded in the participant’s source documents and initialed by the investigator. |
| 12. | Historical HIV-1 genotyping result at screening for children aged ≥2 to <6 years |
| | (and for children aged ≥6 to <12 years if a historical HIV-1 genotyping result is |
| | available at screening) must demonstrate sensitivity to RPV and to the selected |
| | background ARVs. |
| 13. | Girls are eligible to participate if they are not pregnant and not breastfeeding. |
| 14. | Girls of childbearing potential must have a negative highly sensitive serum β-human |
| | chorionic gonadotropin test at screening. |
| 15. | Heterosexually active girls of childbearing potential must practice a highly effective |
| | method of contraception (failure rate of <1% per year when used consistently and |
| | correctly) and agree to remain on a highly effective method while receiving study |
| | treatment and for at least 30 days after last RPV intake. |
| 16. | Heterosexually active boys must practice a highly effective method of contraception |
| | (failure rate of <1% per year when used consistently and correctly) and agree to |
| | remain on a highly effective method while receiving study treatment and for at least |
| | 30 days after last RPV intake. All HIV-1-infected boys are advised to use a condom |
| | to reduce the risk of transmitting HIV. |
| 17. | Can adhere to the lifestyle restrictions |

### Exclusion Criteria

| 1. | Have previously documented HIV-2 infection. | | | |
|---|---|---|---|---|
| 2. | Have known or suspected acute (primary) HIV-1 infection. | | | |
| 3. | Taken any disallowed concomitant therapies within 4 weeks before the planned first | | | |
| | dose of study intervention. | | | |
| 4. | A positive HLA-B*5701 test at screening (when the investigator considers ABC in the | | | |
| | background regimen). In case of a positive test, ABC cannot be administered, but | | | |
| | instead, the investigator can select another ARV in the background regimen. | | | |
| | HLA-B*5701 testing is not required for participants with prior | | | |
| | documented negative results. | | | |
| 5. | Any current or history of adrenal disorder. | | | |
| 6. | Any active clinically significant diseases (eg, pancreatitis, cardiac dysfunction, active | | | |
| | and significant psychiatric disorders, clinical suspicion of adrenal insufficiency, and | | | |
| | hepatic impairment) or findings at screening or medical history that, in the | | | |
| | investigator’s opinion, would compromise the outcome of the study. | | | |
| 7. | A history of virologic failure to ARVs with or without availability of an HIV-1 | | | |
| | genotype result at the time of failure. | | | |
| 8. | Documented genotypic evidence of resistance to RPV or to the selected background | | | |
| | ARVs from historical data available in the source documents (ie, at least 1 NNRTI | | | |
| | RAM from the following list compiled on the basis of the list of the International | | | |
| | Antiviral Society United States of America [IAS-USA] NNRTI RAMs and other | | | |
| | relevant publications). | | | |
| | A098G | V106M | Y181C | G190S |
| | L100I | V108I | Y181I | G190T |
| | K101E | E138A | Y181V | P225H |
| | K101P | E138G | Y188C | F227C |
| | K101Q | E138K | Y188H | M230I |
| | K103H | E138Q | Y188L | M230L |
| | K103N | E138R | G190A | P236L |
| | K103S | V179E | G190C | K238N |
| | K103T | V179D | G190E | K238T |
| | V106A | V179T | G190Q | Y318F |

| | |
|---|---|
| 9. | A known clinically significant allergy, hypersensitivity, or intolerance to RPV or its |
| | excipients or to the selected background ARVs. |
| 10. | 10.1 Received an investigational intervention (including investigational vaccines) |
| | containing an active substance or used an invasive investigational medical device |
| | within 90 days before the planned first dose of study intervention. |
| 11. | Enrolled in clinical studies that include any blood sampling with a volume >50 mL |
| | taken within 6 months before the planned first administration of RPV, specimen |
| | collection, or other interventional procedure. Concurrent participation in |
| | non-interventional observational studies is allowed as long as there is no impact |
| | on the objectives of this study. Data collected in this study can be reported |
| | in the observational study. |
| 12. | Any condition (including but not limited to the abuse of alcohol or drugs |
| | [eg, barbiturates, opiates, cocaine, cannabinoids, amphetamines, and benzodiazepines]) |
| | for which, in the opinion of the investigator, participation would not be in the best |
| | interest of the participant (eg, compromise the well-being) or that could prevent, limit, |
| | or confound the protocol-specified assessments. |
| 13. | A life expectancy of less than 6 months. |
| 14. | Any currently active AIDS-defining illness or Stage-3-defining Opportunistic Illnesses |
| | in HIV Infection (cut-off for Stage-3 illnesses is 6 years of age per criteria from 2014). |
| 15. | Any grade 3/4 laboratory abnormality at screening according to the Division of AIDS |
| | (DAIDS) grading table, except for a selection of abnormalities: |
| | a) grade 3 absolute neutrophil count |
| | b) grade 3 platelets |
| | c) grade 3 glucose elevation in diabetics |
| | d) asymptomatic grade 3 pancreatic amylase elevation |
| | e) asymptomatic grade 3 triglyceride/cholesterol/glucose elevation |
| | f) asymptomatic grade 4 triglyceride elevation |
| 16. | Active tuberculosis or being treated for tuberculosis with rifamycins at screening. |
| 17. | The following ECG findings at screening, if judged clinically significant by the |
| | investigator: abnormal pulse rate and QRS intervals; rhythm abnormalities; evidence |
| | of acute ischemic changes. |
| 18. | One or more of the following risk factors for QTc prolongation: |
| | a) a confirmed prolongation of QT/QTc interval, eg, repeated demonstration of |
| | QT interval corrected for heart rate according to Bazett’s formula (QTcB) or |
| | Fridericia’s formula (QTcF) ≥450 ms in the screening ECG. |
| | b) pathological Q-waves (defined as Q-wave >40 ms or depth >0.4-0.5 mV). |
| | c) evidence of ventricular pre-excitation. |
| | d) electrocardiographic evidence of complete or incomplete left bundle branch |
| | block or complete or clinically significant incomplete right bundle branch block. |
| | e) evidence of second or third degree heart block. |
| | f) intraventricular conduction delay with QRS duration >90 ms. |
| | g) bradycardia as defined by sinus rate <50 bpm. |
| | h) personal or family history of long QT syndrome. |
| | i) personal history of cardiac disease (including congenital heart disease), |
| | symptomatic or asymptomatic arrhythmias, with the exception of sinus arrhythmia. |
| | j) risk factors for Torsade de Pointes (TdP) (eg, heart failure, hypokalemia, and |
| | hypomagnesemia). |
| 19. | Acute clinical hepatitis at screening. |

### Background Regimen

The investigator-selected ARVs, including but not limited to N(t)RTIs (eg, AZT, ABC, TAF, or TDF in combination with FTC or 3TC), whichever are approved and marketed or considered local standard of care for children aged ≥2 to <12 years in a particular country, will be given as the coformulation or as the separate components according to local availability and use in the country (eg, Combivir® or 3TC/AZT, Epzicom®/Kivexa® or ABC/3TC, Truvada® or FTC/TDF). Integrase inhibitors (eg, DTG or raltegravir) can also be administered in combination with RPV, as appropriate. The dual combination of DTG and RPV is currently only approved in adults. Protease inhibitors and ARVs requiring a PK booster, however, are disallowed from baseline onwards.

The selected background ARVs will be used in doses that are specified in the individual package inserts or for which sufficient supporting data are available for use in this age group. Applicable procedures and guidance based on package inserts should be respected (eg, in case of missed doses). The intake of the background ARVs will be according to the locally applicable procedures and package inserts, but preferably at the same time as RPV for ARVs with a once daily regimen. For ARVs with a twice daily regimen, one of the doses will be preferably taken together with RPV and the other dose will be taken according to the package insert. All ARVs should be started on the same day (ie, Day 1). For storage conditions of background ARVs, consult the respective package inserts.

Temporary discontinuation of all ARVs during the study intervention phase will be allowed only in the event of suspected toxicity.

For those participants who do not tolerate the selected background ARVs, switching to alternative ARVs (branded versions [ie, generics with tentative United States FDA approval and/or WHO prequalified drugs], or if not available, generic drugs approved by the local health authorities or procured by the UN international organizations upon approval by the sponsor) is allowed for some predefined toxicities.

### Efficacy Assessments

Blood samples for the determination of plasma HIV-1 RNA viral load to assess antiviral activity and samples for the determination of CD4⁺ cell counts (absolute and percentage relative to total lymphocytes) will be taken.

Plasma viral load levels will be measured at a central lab using a standardized HIV-1 viral load assay as the concentration of HIV-1 RNA in plasma. CD4⁺ cell counts will be measured at a central lab via flow cytometry. Specimen preparation procedures will be defined in the laboratory manual.

Changes from baseline in plasma viral load or in CD4⁺ cell counts (either increases or decreases) will not be reported as (S)AEs.

### Safety Assessments

Safety and tolerability will be evaluated throughout the study from signing of the ICF onwards until the last study-related activity.

Adverse events will be reported and followed by the investigator Adverse events of interest are based on their relevance in the target population, their known association with other ARVs, and/or their potential importance demonstrated by nonclinical and clinical data with RPV, and include endocrine events of interest, potential QTc interval prolonging events of interest, hepatic events of interest, neuropsychiatric events of interest, and skin events of interest.

Any clinically relevant changes occurring during the study should be recorded in the AE section of the CRF. Any clinically significant abnormalities persisting at the last study visit will be followed by the investigator until resolution or until a clinically stable condition is reached.

Venous blood samples of approximately 1 mL will be collected for measurement of plasma concentrations of RPV at the predetermined time points.

### Analytical Procedures

Plasma PK samples will be analyzed to determine concentrations of RPV using a validated, specific, and sensitive liquid chromatography-mass spectrometry/mass spectrometry method by or under the supervision of the sponsor.

If needed, some plasma samples may be analyzed to document the presence of circulating metabolites using a qualified research method.

### Pharmacokinetic Parameters and Evaluations

Based on the individual plasma concentration-time data, using the actual dose taken and the actual PK sampling times, the following PK parameters of RPV will be derived:

C₀ₕ, Cₘᵢₙ, Cₘₐₓ, C_{ss,av}, tₘₐₓ, AUC₂₄ₕ, CL/F, Vₛₛ/F, and FI

Other PK parameters may be estimated for exploration of the data, as appropriate.

For the PK parameters, definitions and methods of calculation are:

| | |
|---|---|
| C₀ₕ: | predose plasma concentration |
| Cₘᵢₙ: | minimum observed plasma concentration |
| Cₘₐₓ: | maximum observed plasma concentration |
| C_{ss,av}: | average plasma concentration at steady state |
| tₘₐₓ: | time to reach the maximum observed plasma concentration |
| AUC₂₄ₕ: | area under the plasma concentration-time curve from time |
| | of administration up to 24 hours postdose |
| CL/F: | total apparent clearance at steady state, calculated |
| | by dose/AUC₂₄ₕ |
| V_{SS}/F: | apparent volume of distribution at steady state |
| FI: | fluctuation index |

### Efficacy Analyses

### Plasma Viral Load

An outcome analysis (ie, proportion of participants with a plasma viral load <50 and <400 HIV-1 RNA copies/mL) will be performed using Snapshot approach. The Snapshot analysis is based on the last observed plasma viral load data within the visit window (ie, Weeks 24 and 48). The proportion of participants with virologic failure (ie, HIV-1 RNA ≥50 and ≥400 copies/mL) per Snapshot approach will be provided. Participants who switched ARVs for tolerability reasons not allowed per protocol will be considered as virologic failures for this Snapshot approach. Proportions will be expressed as percentages with Clopper Pearson 95% CI at each time point.

Time-to-event data (ie, time to loss of virologic response) will be graphically presented by means of Kaplan-Meier curves.

### CD4⁺ Cell Count

The analysis will be based on observed values and on imputed values using NC=F, ie, participants who prematurely discontinued the study will have their CD4⁺ cell count following discontinuation imputed with the baseline value (resulting in a change of 0), and will have last-observation-carried-forward imputation for intermediate missing values.

Actual data and changes from baseline will be descriptively and graphically presented.

### Safety Analyses

### Adverse Events/HIV-Related Events

The verbatim terms used in the CRF by investigators to identify AEs will be coded using the Medical Dictionary for Regulatory Activities. Treatment-emergent AEs (including HIV-related events) are AEs with onset during the intervention phase or that are a consequence of a pre-existing condition that has worsened since baseline. All reported AEs will be included in the analysis. For each treatment-emergent AE/HIV-related event, the percentage of participants who experience at least 1 occurrence of the given event will be tabulated per study phase (ie, screening phase, intervention phase, and follow-up). Separate tabulations will be made by severity and relationship to the study intervention, as appropriate.

Summaries, listings, datasets, or participant narratives may be provided, as appropriate, for those participants who die, who discontinue study intervention due to an AE, or who experience a grade 3/4 AE, an AE of special interest, or an SAE.

### Clinical Laboratory Tests

Laboratory data will be summarized by type of laboratory test. Descriptive statistics will be calculated for each laboratory analyte at baseline and for observed values and changes from baseline at each scheduled time point. Descriptive statistics include number of observations (n), mean, standard deviation (SD), median, minimum, and maximum.

Frequency tabulations of the changes from baseline will be presented in pre- versus post-intervention cross-tabulations (with classes for below, within, and above normal ranges). For the tests available, laboratory abnormalities will be determined using the DAIDS grading table. Frequency tabulations of worst abnormality grade after baseline will be generated. As appropriate, frequency tabulations and listings will be provided for participants who develop a grade 3/4 laboratory abnormality.

Descriptive statistics of the actual values and changes from baseline of the endocrine assessments (cortisol, follicle-stimulating hormone [FSH], luteinizing hormone [LH], androstenedione, testosterone, and dehydroepiandrosterone sulfate [DHEAS]) will be generated.

Descriptive statistics of the actual values and changes from baseline of the endocrine assessments (cortisol and 17-hydroxyprogesterone) at 60 minutes after ACTH stimulation will be presented. In addition, the proportion of participants with cortisol values <500 nmol/L (18.1 μg/dL) before and 60 minutes after ACTH stimulation will be tabulated.

### Electrocardiogram

Descriptive statistics of ECG values and changes from baseline will be summarized at each scheduled time point. The ECG parameters analyzed are heart rate, PR interval, QRS interval, RR interval, QT interval, QTcB, and QTcF. Descriptive statistics include number of observations (n), mean, SD, median, minimum, and maximum. Frequency tabulations of the abnormalities will be made.

### Vital Signs

Descriptive statistics of pulse rate and blood pressure (systolic and diastolic) (supine and standing) values and changes from baseline will be summarized at each scheduled time point. Descriptive statistics include number of observations (n), mean, SD, median, minimum, and maximum. The percentage of participants with values beyond clinically important limits will be summarized at each time point.

### Physical Examination

Physical examination findings will be summarized at each scheduled time point per body system. Physical examination abnormalities will be listed.

Growth will be followed regularly and evaluated consistently using standardized growth charts. Descriptive statistics of height, height-for-age, weight, weight-for-age, body mass index (BMI), and BMI-for-age will be calculated at baseline and for observed values and changes from baseline at each scheduled time point. Descriptive statistics include number of observations (n), mean, SD, median, minimum, and maximum.

Tanner stage (for pubic hair and genitalia/breasts) will be cross-tabulated versus baseline by age. In addition, in girls, the occurrence of first menses during treatment will also be cross-tabulated versus baseline, and the date of menarche will be listed.

### Example 2

A Phase II, open label, single arm trial to evaluate the pharmacokinetics, safety, tolerability, and antiviral activity of rilpivirine (TMC278) in antiretroviral-naive HIV-1 infected adolescents and children aged ≥6 to <18 years.

The purpose of this study is to evaluate the pharmacokinetics, safety and antiviral activity of rilpivirine (TMC278) 25 milligram (mg) or adjusted dose once daily in combination with an investigator-selected background regimen containing 2 nucleoside/nucleotide reverse transcriptase inhibitors (N[t]RTIs) (zidovudine [AZT], abacavir [ABC], or tenofovir disoproxil fumarate [TDF] in combination with lamivudine [3TC] or emtricitabine [FTC] in antiretroviral (ARV) treatment-naïve adolescents and children aged greater than or equal to (>=) 6 to less than (<) 18 years.

### Detailed Description

This is a Phase II, open-label (all people involved know the identity of the assigned drug) and single arm study. The study will consist of a screening period of maximum 8 weeks, an initial treatment period of 48 weeks, a post week 48 treatment extension period of 4 years (Cohort 1 only), and a 4 week follow-up (cohort 2 only) period. Participants who withdraw from the trial on or before the Week 48 visit or subjects with ongoing (serious) adverse events ([S]AEs), laboratory abnormalities, or viral load increase at the last on-treatment visit in the extension, will be seen for a follow-up visit 4 weeks later. The initial 48-week treatment period will be structured into 2 age Cohorts; Cohort 1 (Aged greater than or equal to [>=] 12 to less than [<] 18 years) and Cohort 2 (Children Aged >=6 to <12 years). The trial is designed to evaluate the steady-state pharmacokinetic (PK) profile (based on intensive PK analysis) and the short-term safety and antiviral activity of rilpivirine (RPV). Participants will receive RPV 25 milligram (mg), or weight-adjusted dose orally once daily for 240 weeks when administered in combination with 2 nucleoside/nucleotide reverse transcriptase inhibitors (NRTIs). The trial will also evaluate long-term (48 weeks and 240 weeks [Cohort 1]) safety, efficacy, and pharmacokinetics of rilpivirine in combination with the background regimen of 2 NRTIs. Patients safety will be monitored throughout the study and during the follow up visits.

### Arms and Interventions

| Arm | Intervention/treatment |
|---|---|
| Experimental Rilpivirine (TMC278) | Drug Rilpivirine |
| The patients received rilpivirine with 2 | Patients will receive rilpivirine (RPV) tablet |
| nucleoside/nucleotide reverse transcriptase | 25 milligram dose or an adjusted dose orally |
| inhibitors (NRTIs) as a background regimen | once daily in Cohort 1 (adolescents aged >= |
| in cohort 1 [aged greater than or equal to (>=) | 12 to <18 years) up to 240 weeks Patients |
| 12 to less than (<) 18 years] for up to 240 | will receive RPV weight-adjusted dose |
| weeks which is already completed and | orally once daily in Cohort 2 (children aged >= |
| recruitment closed and will receive this | 6 to <12 years) or 25 mg once daily for up |
| treatment in cohort 2 (children aged >= 6 | to 48 weeks |
| to <12 years) for up to 48 weeks The NRTIs | Drug: Zidovudine |
| include zidovudine, abacavir, or tenofovir | Type = exact, form = appropriate pediatric |
| disoproxil fumarate in combination with | formulation, unit = mg, route = oral. The |
| lamivudine or emtricitabine | patients may receive this selected NRTI |
| | together with another NRTI once daily for |
| | up to 48 weeks (Cohort 2) and 240 weeks |
| | (Cohort 1). |
| | Drug: Abacavir |
| | Type = exact, form = appropriate pediatric |
| | formulation, unit = mg, route = oral. The |
| | patients may receive this selected NRTI |
| | together with another NRTI once daily for |
| | up to 48 weeks (Cohort 2) and 240 weeks |
| | (Cohort 1). |
| | Drug: Tenofovir disoproxil fumarate |
| | Type = exact, form = appropriate pediatric |
| | formulation, unit = mg, route = oral. The |
| | patients may receive this selected NRTI |
| | together with another NRTI once daily for |
| | 240 weeks (Cohort 1). |
| | Drug: Lamivudine |
| | Type = exact, form = appropriate pediatric |
| | formulation, unit = mg, route = oral. The |
| | patients may receive this selected NRTI |
| | together with another NRTI once daily for |
| | up to 48 weeks (Cohort 2) and 240 weeks |
| | (Cohort 1). |
| | Drug: Emtricitabine |
| | Type = exact, form = appropriate pediatric |
| | formulation, unit = mg, route = oral. The |
| | patients may receive this selected NRTI |
| | together with another NRTI once daily for |
| | up to 48 weeks (Cohort 2) and 240 weeks |
| | (Cohort 1). |

### Primary Outcome Measures

1. Pharmacokinetics (PK) of Rilpivirine (TMC278) as Measured by Maximum Plasma Concentration (Cmax) [Time Frame: Up to 48 weeks]

2. Pharmacokinetics of Rilpivirine as Measured by Area Under the Plasma Concentration Curve (AUC24) [Time Frame: Up to 48 weeks]

AUC24 is defined area under the plasma concentration time curve from 0 to 24 hours post dosing of rilpivirine.

### Secondary Outcome Measures

1. Number of Patients with Adverse Events [Time Frame: Up to 244 weeks (Cohort 1 only) (including 4 week follow up visit)]

Safety measures include adverse events, vital signs, physical examination, hematology, biochemistry and electrocardiogram.

2. Percentage of Participants With Plasma Human Immunodeficiency Virus-1 (HIV-1) Ribonucleic Acid (RNA) level Less Than (<) 50 Copies/mL Defined by the Time to Loss of Virologic Response (TLOVR) Algorithm [Time Frame: Week 48 and Week 240 (Cohort 1 only)]

Time to loss of virologic response algorithm (TLOVR) requires sustained HIV-1 RNA <50 copies/mL; confirmed HIV-1 RNA more than or equal to (>=) 50 copies/mL is considered as non-response (rebound); participant is considered non-responder after permanent discontinuation.

3. Percentage of Participants with Plasma HIV-1 RNA <50 Copies/mL by FDA Snapshot Approach [Time Frame: Week 48 and Week 240 (Cohort 1 only)]

FDA Snapshot Approach is based on the last observed viral load data within the Week 48 window: virologic response is defined as HIV-1 RNA <50 copies/mL (observed case); missing HIV-1 RNA is considered as non-response.

4. Evolution of viral genotype and phenotype [Time Frame: Up to 48 Weeks and 240 Weeks (Cohort 1 only)]

Blood samples will be collected for the determination of HIV-1 genotype and phenotype by the Protocol Virologist based on plasma viral load.

5. Treatment adherence as measured by the Study Adherence Questionnaire [Time Frame: Up to 48 Weeks and 240 Weeks (Cohort 1 only)]

This endpoint is measured by Study Adherence Questionnaire for children and teenagers. The adherence questionnaire should be completed by the patient. The questionnaire includes questions about the medicine, it's color and dosage.

6. Change in Cluster of Differentiation (CD4+ ) cells [Time Frame: Week 48 and Week 240 (Cohort 1 only)]

Change in the CD4+ cells will evaluate immunologic changes at Week 48 and Week 240 of treatment with rilpivirine.

### Eligibility Criteria

Ages eligible for Study : 6 Years to 17 Years (Child)

### Sexes Eligible for Study: All

### Accepts Healthy Volunteers: No

### Inclusion Criteria

Has documented human immuno deficiency virus (HIV-1) infection
Patients who meet the following criteria; a) Cohort 1: Patients Aged greater than or equal to (>=) 12 to less than (<) 18 years, weight is >=32 kilogram (kg), b) Cohort 2; Aged >=6 to <12 years, weight is >=17 kg
Must have HIV-1 plasma viral load at screening greater than or equal to 500 HIV-1 ribonucleic acid (RNA) copies/mL
Have not received treatment with a therapeutic HIV vaccine or an HIV drug with the exception of a single dose of nevirapine (NVP) (Cohort 1 and Cohort 2) or up to 6 weeks of zidovudine (AZT) use (Cohort 2 only) prior to screening to prevent mother-to-child transmission (MTCT)
In the judgment of the investigator, it is appropriate to initiate antiretroviral therapy (ARV) therapy based on a patient's medical condition and taking into account guidelines for the treatment of HIV-1 infection in children of this age group

### Exclusion Criteria:

Any previous use of ARVs with the exception of single dose NVP (Cohort 1 and Cohort 2) or up to 6 weeks of AZT (Cohort 2 only) to prevent MTCT
Plasma viral load at screening greater than 100,000 HIV-1 RNA copies/mL
Documented genotypic evidence of non-nucleoside reverse transcriptase inhibitor (NNRTI) resistance at screening or from historical data available in the source documents
Use of disallowed concomitant therapy from 4 weeks prior to the baseline visit
Patient has any currently active Acquired Immunodeficiency Syndrome (AIDS) defining illness
Patient has active tuberculosis and/or is being treated for tuberculosis at screening
Personal history of cardiac disease (including congenital heart disease), or symptomatic arrhythmias, with the exception of sinus arrhythmia; personal history of asymptomatic arrhythmias is excluded if the asymptomatic arrhythmia is clinically significant in the opinion of the investigator
Preliminary analysis data are provided in the below table.

PK Parameters (Intensive PK Assessment) of RPV After
Multiple Dose Administration of RPV 25 mg qd in Adolescents
(Cohort 1) and in Children ≥6-<12 years (Cohort 2)

| PK | | | | |
|---|---|---|---|---|
| Parameter | | | | |
| (mean ± SD, | | Children* | Children* | |
| tₘₐₓ: median | | all | ≥25 kg | |
| [range]) | Adolescents | bodyweights | only | Adults** |
| N | 23 | 10 | 5 | 44 |
| C_{0h,} ng/mL | 80.8 ± 40.4 | 118 ± 61.5 | 104 ± 57 | 67.6 ± 39.0 |
| C_{min,} ng/mL | 57.1 ± 26.3 | 77 ± 38.4 | 74 ± 29.7 | 53.7 ± 28.3 |
| C_{max,} ng/mL | 109 ± 38.0 | 182 ± 110 | 136 ± 46.1 | 134 ± 72.0 |
| tₘₐₓ, h | 5.00 (2.00-9.03) | 4.50 (1.97-9.00) | 4.00 (1.97-9.00) | 4.00 (1.00-12.00) |
| AUC_{24h,} | 1872 ± 717 | 3017 ± 1606 | 2452 ± 735 | 2005 ± 970 |
| ng*h/mL | | | | |

*Preliminary analysis for children
Target GM RPV AUC₂₄ₕ: 1462-2673 ng*h/mL
**PK Parameters for Adults from pooled ECHO/THRIVE

1. A method of treating a pediatric subject infected with an HIV virus comprising administering to the subject:
25 mg or less of a non-nucleoside reverse transcriptase inhibitor that is rilpivirine, or an equivalent amount of a pharmaceutically acceptable salt of rilpivirine, once daily;
wherein the subject weighs 11 kg or more; is treatment experienced; and was administered a first anti-retroviral regimen that has been discontinued;
and
wherein the subject exhibits a viral load of less than or equal to 50 copies of HIV virus particles per mL of blood plasma (≤50 c/mL) after at least 24 weeks of the once-daily administration of the rilpivirine, or equivalent amount of the pharmaceutically acceptable salt of rilpivirine.

2. The method of claim 1, wherein the subject is administered a pharmaceutically acceptable salt of rilpivirine.

3. The method of claim 1, wherein the pharmaceutically acceptable salt of rilpivirine is the hydrochloride salt of rilpivirine.

4. The method of claim 1, further comprising administering to the pediatric subject dolutegravir, tenofovir, tenofovir disoproxil fumarate, tenofovir alafenamide, emtricitabine, or a combination thereof

5. The method of claim 1, wherein the pediatric subject is ≥2 to <12 years.

6. The method of claim 1, wherein the pediatric subject has a body weight of <25 kg.

7. The method of claim 1, wherein the pediatric subject has a body weight of ≤70 kg.

8. The method of claim 1, wherein the pediatric subject exhibits a viral load of less than or equal to 50 copies of HIV virus particles per mL of blood plasma (≤50 c/mL) prior to the once-daily administration of the rilpivirine, or equivalent amount of the pharmaceutically acceptable salt of rilpivirine.

9. The method of claim 1, wherein the HIV virus is HIV-1.

10. The method of claim 1, wherein the 25 mg or less of a non-nucleoside reverse transcriptase inhibitor that is rilpivirine, or the equivalent amount of the pharmaceutically acceptable salt of rilpivirine, is administered as a single unit dosage form.

11. The method of claim 1, wherein the 25 mg or less of a non-nucleoside reverse transcriptase inhibitor that is rilpivirine, or the equivalent amount of the pharmaceutically acceptable salt of rilpivirine, is administered in multiple single unit dosage forms.

12. The method of claim 1, wherein the subject exhibits a viral load of less than or equal to 50 copies of HIV-1 virus particles per mL of blood plasma (≤50 c/mL) after at least 48 weeks of once daily administration of the rilpivirine, or the pharmaceutically acceptable salt of rilpivirine.
